(19) United States
(12) Patent Application Publication
(10) Pub. No.: US 2026/0115429 A1
(43) Pub. Date: Apr. 30, 2026

(54) Tactile-Feedback System and Method for Guidewire-Containing Medical Devices

(71) Applicant: Bard Access Systems, Inc., Salt Lake City, UT (US)

(72) Inventors: John Stokes, Ogden, UT (US); Kristin N. Rominger, South Jordan, UT (US)

(21) Appl. No.: 18/932,901

(22) Filed: Oct. 31, 2024

(51) Int. Cl. A61M 25/09

(52) U.S. Cl. CPC A61M 25/09041; A61M 2025/09141

(57) ABSTRACT

A tactile-feedback system and method for guidewire-containing medical devices facilitates locating a guidewire within both a medical device and a blood vessel of a patient. In an example, a tactile-feedback system for a guidewire-containing medical device includes a guidewire and a rigid member in vibratory communication with a handholdable portion of the medical device. The guidewire includes interactive features along its length. The interactive features of the guidewire interact with the rigid member to produce user-perceptible vibrations in the handholdable portion of the medical device as the guidewire moves against the rigid member while being advanced out of the medical device or retracted into the device. The user-perceptible vibrations provide tactile feedback to a user of the medical device indicating a status of the guidewire with respect to a patient or the medical device.

### BACKGROUND

Advancing a guidewire from a medical device into a patient after a venipuncture can be challenging due to several factors. One challenging factor is determining whether the guidewire is properly located within the medical device prior to advancing the guidewire from the medical device into the patient. This uncertainty can arise because any resistance on the guidewire within the medical device is subtle, which makes it hard to distinguish between portions of the medical device. Another challenging factor is determining whether the guidewire is properly located within the patient upon advancing the guidewire from the medical device into the patient. Indeed, without ultrasound or fluoroscopic guidance, it can be difficult to determine how far the guidewire has advanced within a blood vessel of the patient or even if it has inadvertently exited the blood vessel in an instance of backwalling. In view of the foregoing, a solution is needed for locating a guidewire within both a medical device and a blood vessel of a patient.

Disclosed herein is a tactile-feedback system and method for guidewire-containing medical devices.

### SUMMARY

Disclosed herein is a tactile-feedback system for a guidewire-containing medical device including a guidewire and a rigid member in vibratory communication with a handholdable portion of the medical device. The guidewire includes interactive features along its length. The interactive features of the guidewire interact with the rigid member to produce user-perceptible vibrations in the guidewire or the handholdable portion of the medical device as the guidewire moves against the rigid member while being advanced out of the medical device or retracted into the device. The user-perceptible vibrations provide tactile feedback to a user of the medical device indicating a status of the guidewire with respect to a patient or the medical device.

In some embodiments, the guidewire moves over the rigid member. With respect to being over the rigid member, the guidewire is between the rigid member and the user of the medical device from a perspective of the user looking down onto the medical device.

In some embodiments, the guidewire moves under the rigid member. With respect to being under the rigid member, the rigid member is between the guidewire and the user of the medical device from a perspective of the user looking down onto the medical device.

In some embodiments, the guidewire moves through a through hole of the rigid member.

In some embodiments, the rigid member is a metal insert disposed in a housing of the medical device or a structural component associated with the housing.

In some embodiments, the rigid member is a metal rod or plate.

In some embodiments, the status of the guidewire with respect to the patient is a depth to which the guidewire is inserted into the patient.

In some embodiments, the status of the guidewire with respect to the medical device is whether a distal tip of the guidewire is within the medical device or outside the medical device.

In some embodiments, the interactive features of the guidewire include differences in guidewire diameter, surface features, surface characteristics, composition, construction, or some combination thereof.

In some embodiments, the guidewire diameter changes from one of a larger diameter or a smaller diameter to the other of the larger diameter or the smaller diameter to indicate the status of the guidewire with respect to the patient or the medical device.

In some embodiments, the guidewire surface features change from one of a presence or a lack of the guidewire surface features to the other of the presence or the lack of the guidewire surface features to indicate the status of the guidewire with respect to the patient or the medical device.

In some embodiments, the guidewire surface features are ridges, grooves, or some combination thereof.

In some embodiments, the guidewire surface characteristics include surface roughness that changes from one degree of the surface roughness to another degree of the surface roughness to indicate the status of the guidewire with respect to the patient or the medical device.

In some embodiments, the guidewire construction changes from one of a bare-wire portion, a round-wound construction, a ground-wound construction, or a flat-wound potion of the guidewire to another of the bare-wire portion, the round-wound construction, the ground-wound construction, or the flat-wound potion of the guidewire to indicate the status of the guidewire with respect to the patient or the medical device.

In some embodiments, the medical device is a catheter assembly.

In some embodiments, the medical device is a guidewire-management device.

Also disclosed herein is a method of using a guidewire-containing medical device with a tactile-feedback system. The method includes, in some embodiments, obtaining the medical device. The tactile-feedback system includes a guidewire and a rigid member in vibratory communication with a handholdable portion of the medical device. The guidewire includes interactive features along its length. The interactive features of the guidewire interact with the rigid member to produce user-perceptible vibrations in the handholdable portion of the medical device as the guidewire moves against the rigid member. The method also includes advancing the guidewire out of the medical device or retracting the guidewire into the medical device, thereby providing tactile feedback to a user of the medical device indicating a status of the guidewire with respect to a patient or the medical device.

In some embodiments, advancing the guidewire out of the medical device or retracting the guidewire into the medical device moves the guidewire over the rigid member. With respect to being over the rigid member, the guidewire is between the rigid member and the user of the medical device from a perspective of the user looking down onto the medical device.

In some embodiments, the rigid member is a metal rod or plate disposed in a housing of the medical device or a structural component associated with the housing.

In some embodiments, the status of the guidewire with respect to the patient is a depth to which the guidewire is inserted into the patient, and the status of the guidewire with respect to the medical device is whether a distal tip of the guidewire is within the medical device or outside the medical device.

In some embodiments, the interactive feature of the guidewire include differences in guidewire diameter, surface features, surface characteristics, composition, construction, or some combination thereof.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates a catheter assembly as a medical device including a tactile-feedback system in accordance with some embodiments.

FIG. 2 illustrates a top view of the catheter assembly in accordance with some embodiments.

FIG. 3 illustrates a longitudinal cross section of a handholdable portion of the catheter assembly in accordance with some embodiments.

FIG. 4 illustrates retraction of a guidewire into the catheter assembly with tactile feedback in accordance with some embodiments.

FIG. 5 illustrates a guidewire-management device as a medical device including the tactile-feedback system in accordance with some embodiments.

FIG. 6 illustrates advancement of the guidewire out of the guidewire-management device with tactile feedback in accordance with some embodiments.

FIG. 7 illustrates a detailed view of a generic guidewire including interacting features in accordance with some embodiments.

FIG. 8A illustrates a detailed view the interactive features including differences in guidewire surface characteristics in accordance with some embodiments.

FIG. 8B illustrates a detailed view of the interactive features including differences in guidewire diameter in accordance with some embodiments.

FIG. 8C illustrates a detailed view of the interactive features including differences in guidewire construction in accordance with some embodiments.

FIG. 9 illustrates a detailed view of the guidewire including a plurality of interactive features in accordance with some embodiments.

FIG. 10 illustrates a detailed view of the guidewire including another plurality of interactive features in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

“Proximal” is used to indicate a portion, section, piece, element, or the like of a medical device intended to be near or relatively nearer to a clinician when the medical device is used on a patient. For example, a “proximal portion” or “proximal section” of the medical device includes a portion or section of the medical device intended to be near the clinician when the medical device is used on the patient. Likewise, a “proximal length” of the medical device includes a length of the medical device intended to be near the clinician when the medical device is used on the patient. A “proximal end” of the medical device is an end of the medical device intended to be near the clinician when the medical device is used on the patient. The proximal portion, the proximal section, or the proximal length of the medical device need not include the proximal end of the medical device. Indeed, the proximal portion, the proximal section, or the proximal length of the medical device can be short of the proximal end of the medical device. However, the proximal portion, the proximal section, or the proximal length of the medical device can include the proximal end of the medical device. Should context not suggest the proximal portion, the proximal section, or the proximal length of the medical device includes the proximal end of the medical device, or if it is deemed expedient in the following description, “proximal portion,” “proximal section,” or “proximal length” can be modified to indicate such a portion, section, or length includes an end portion, an end section, or an end length of the medical device for a “proximal end portion,” a “proximal end section,” or a “proximal end length” of the medical device, respectively.

“Distal” is used to indicate a portion, section, piece, element, or the like of a medical device intended to be near, relatively nearer, or even in a patient when the medical device is used on the patient. For example, a “distal portion” or “distal section” of the medical device includes a portion or section of the medical device intended to be near, relatively nearer, or even in the patient when the medical device is used on the patient. Likewise, a “distal length” of the medical device includes a length of the medical device intended to be near, relatively nearer, or even in the patient when the medical device is used on the patient. A “distal end” of the medical device is an end of the medical device intended to be near, relatively nearer, or even in the patient when the medical device is used on the patient. The distal portion, the distal section, or the distal length of the medical device need not include the distal end of the medical device. Indeed, the distal portion, the distal section, or the distal length of the medical device can be short of the distal end of the medical device. However, the distal portion, the distal section, or the distal length of the medical device can include the distal end of the medical device. Should context not suggest the distal portion, the distal section, or the distal length of the medical device includes the distal end of the medical device, or if it is deemed expedient in the following description, “distal portion,” “distal section,” or “distal length” can be modified to indicate such a portion, section, or length includes an end portion, an end section, or an end length of the medical device for a “distal end portion,” a “distal end section,” or a “distal end length” of the medical device, respectively.

Unless defined otherwise, all technical and scientific terms used herein have the same meaning as commonly understood by those of ordinary skill in the art.

As set forth above, advancing a guidewire from a medical device into a patient after a venipuncture can be challenging due to several factors. One challenging factor is determining whether the guidewire is properly located within the medical device prior to advancing the guidewire from the medical device into the patient. This uncertainty can arise because any resistance on the guidewire within the medical device is subtle, which makes it hard to distinguish between portions of the medical device. Another challenging factor is determining whether the guidewire is properly located within the patient upon advancing the guidewire from the medical device into the patient. Indeed, without ultrasound or fluoroscopic guidance, it can be difficult to determine how far the guidewire has advanced within a blood vessel of the patient or even if it has inadvertently exited the blood vessel in an instance of backwalling. In view of the foregoing, a solution is needed for locating a guidewire within both a medical device and a blood vessel of a patient.

Disclosed herein is a tactile-feedback system and method for guidewire-containing medical devices.

### Tactile-Feedback System

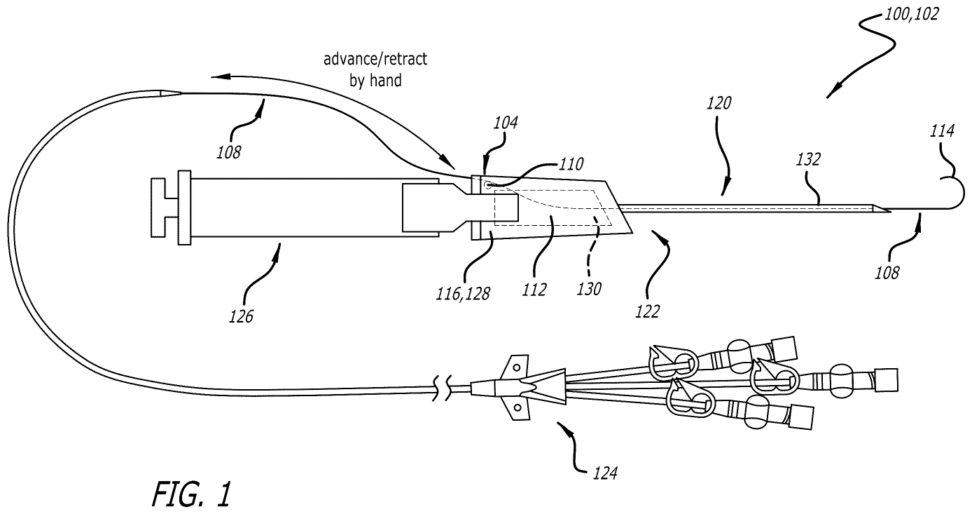

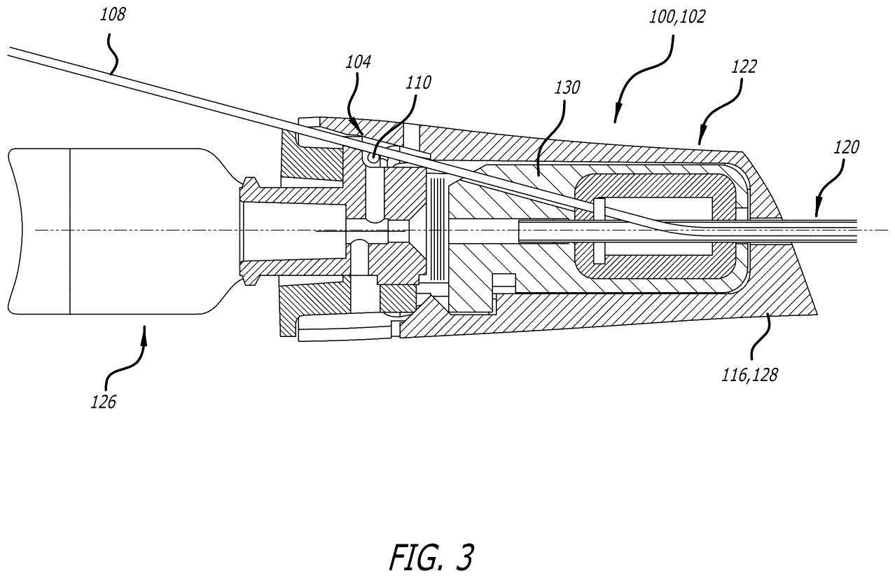

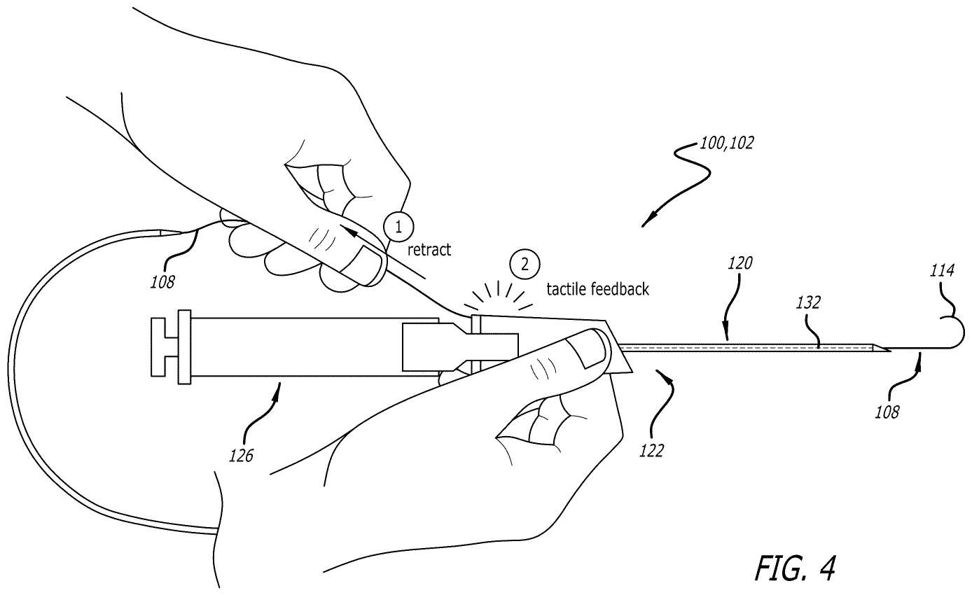

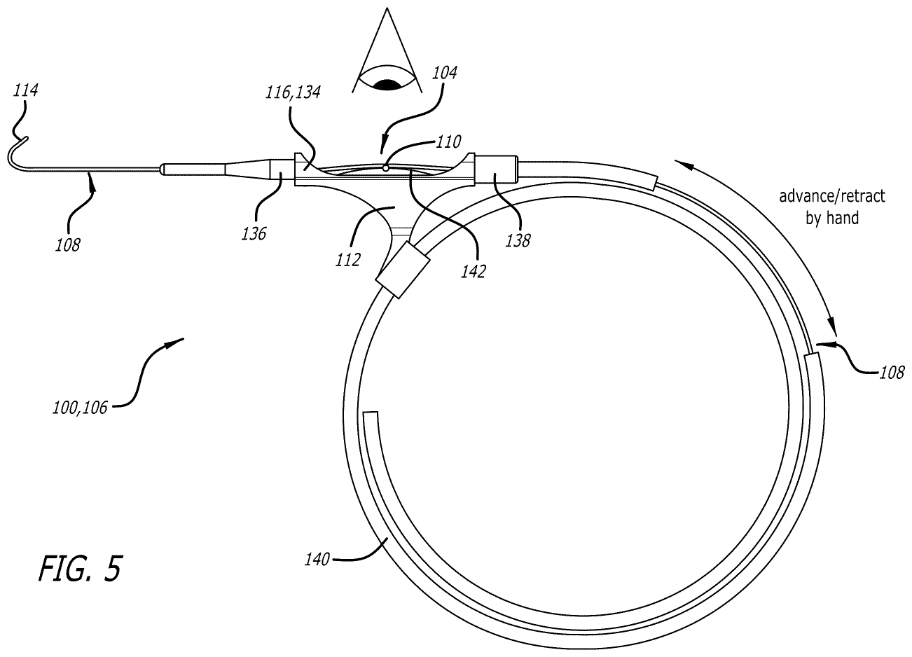

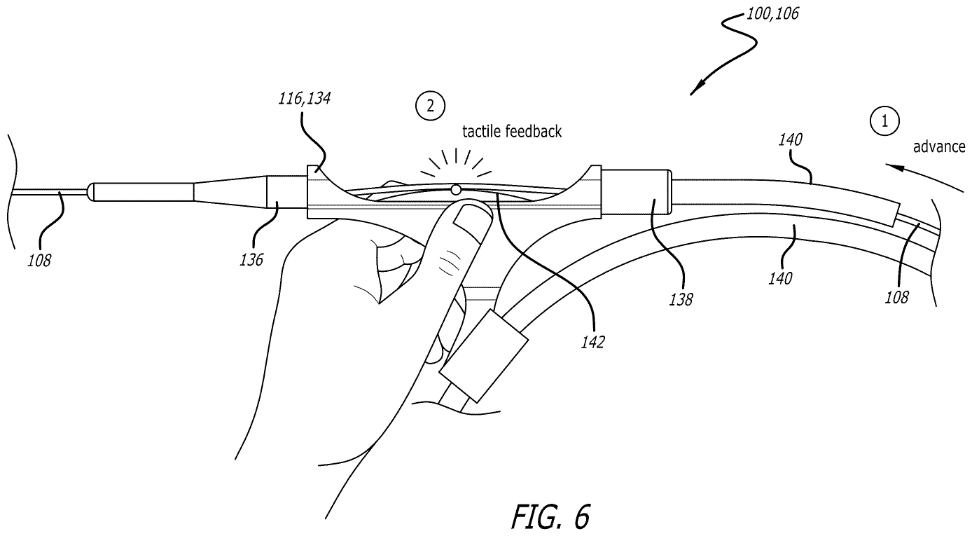

FIGS. 1-4 illustrate a guidewire-containing medical device 100, specifically, a catheter assembly 102, including a tactile-feedback system 104 in accordance with some embodiments. FIGS. 5 and 6 illustrate the medical device 100, albeit, as a guidewire-management device 106, including the tactile-feedback system 104 in accordance with some embodiments. It should be understood that each medical device of the catheter assembly 102 and the guidewire-management device 106 is a species-level example of the medical device 100 including the tactile-feedback system 104. Notably, the tactile-feedback system 104 is initially described in the context of a generic guidewire-containing medical device (i.e., the medical device 100) with references, as expositorily beneficial, to the species-level examples thereof. Subsequently, the species-level examples of the medical device 100, again, the catheter assembly 102 and the guidewire-management device 106, are described in more detail.

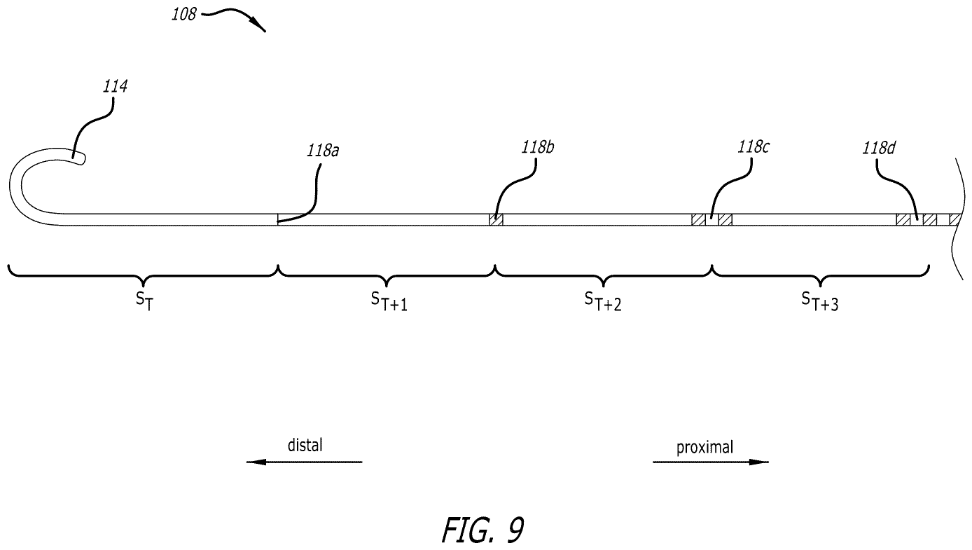

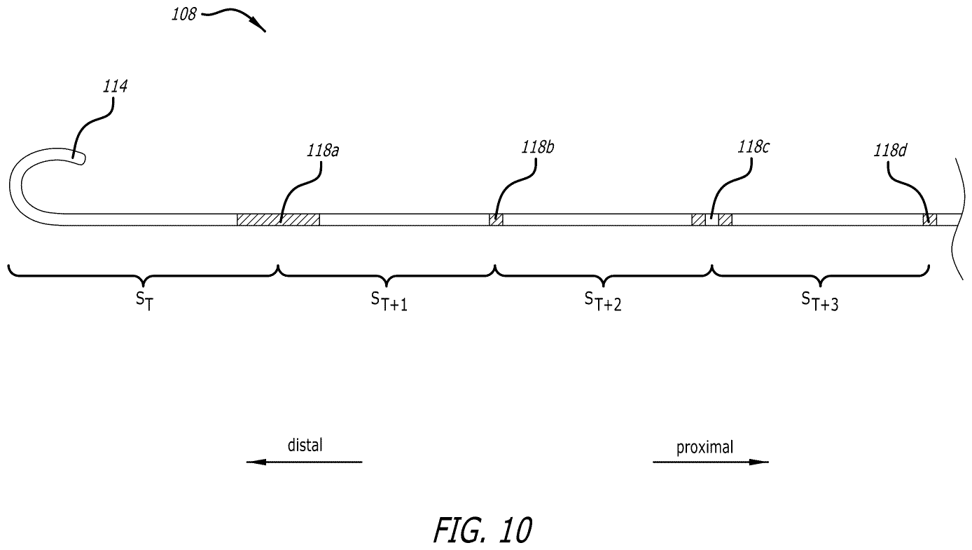

As shown, the tactile-feedback system 104 includes a guidewire 108 and a rigid member 110 in vibratory communication with a handholdable portion 112 of the medical device 100. Functionally, the interactive features 118 of the guidewire 108 set forth below are transitional features that interact with the rigid member 110 to produce, modify (e.g., strengthen or weaken), or stop producing user-perceptible vibrations in the guidewire 108, itself, or the handholdable portion 112 of the medical device 100, which can be felt by a user holding the guidewire 108 or the handholdable portion 112 of the medical device 100, as the guidewire 108 moves against the rigid member 110 while the guidewire 108 is being advanced out of the medical device 100 or retracted into the medical device 100. The user-perceptible vibrations provide tactile feedback characteristic of the interactive features 118 of the guidewire 108 to the user of the medical device 100 that indicate a status of the guidewire 108 with respect to the medical device 100 or a patient upon which the medical device 100 is being used. In an example, the status of the guidewire 108 with respect to the medical device 100 is whether a distal tip 114 of the guidewire 108 is within the medical device 100 or outside the medical device 100. With respect to the latter, the distal tip 114 of the guidewire 108 can be outside the medical device 100, for example, advanced through a distal end of the medical device 100 into the patient or retracted through a proximal end of the medical device 100, thereby disassociating the guidewire 108 from the medical device 100. In another example, the status of the guidewire 108 with respect to the patient is a depth to which the guidewire 108 is inserted into the patient. Notably, indications of the depth to which the guidewire 108 is inserted into the patient, for example, the interactive features 118 set forth below with respect to FIGS. 9 and 10, are also useful in simply indicating whether the guidewire 108 is being advanced or retracted with respect to the medical device 100. Indeed, such indications can be useful for indicating the guidewire 108 is at risk of being retracted through the proximal end of the medical device 100 prior to doing so, particularly when the user-perceptible vibrations are no longer being felt during retraction of the guidewire 108.

Beginning with the rigid member 110, the rigid member 110 can be a rigid insert disposed in a housing 116 of the medical device 100 (e.g., the coupler housing 128 of the catheter assembly 102) or a structural component associated with the housing 116 (e.g., the guidewire support 142 of the guidewire-management device 106). Such an insert can be a beam having any two-dimensional shape in transverse cross section, for example, a cylindrical beam or rod, or the insert can be a plate. Optionally, the insert, whether the foregoing beam or plate, can include a through hole having an inner diameter configured to accommodate up to a greatest outer diameter of the guidewire 108 in embodiments where the through hole of the rigid insert, when present, does not function as a constrictive or abrupt stop for over advancement or over retraction of the guidewire 108. In other words, when the through hole of the rigid insert is present, it can be less than the greatest outer diameter of the guidewire 108, thereby functioning as a constrictive or abrupt stop to prevent over advancement or over retraction of the guidewire 108. Regardless, the rigid insert can be formed of a hard material such as a metal, an alloy, a ceramic, glass, fiberglass, a polymeric material such as a thermoset, carbon fiber, or the like.

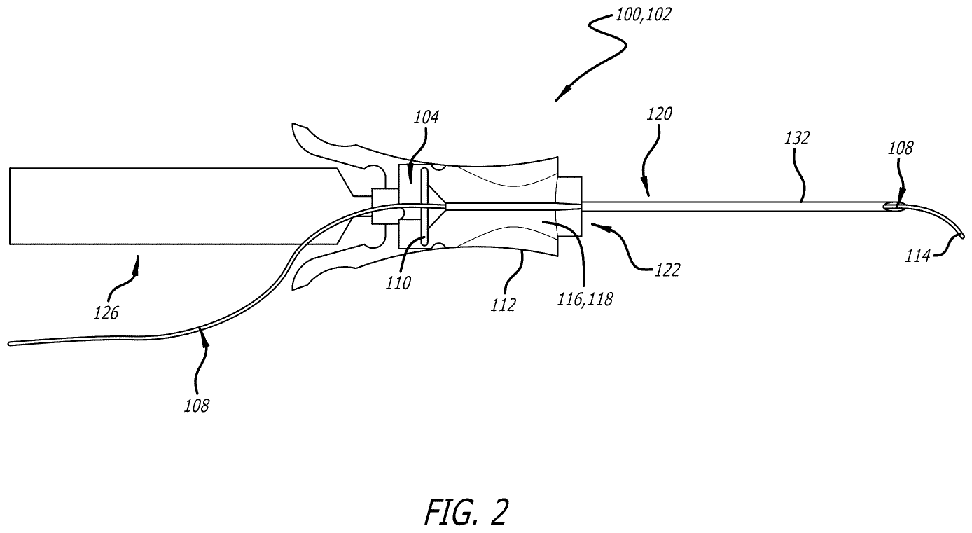

The rigid member 110 can be disposed in the medical device 100 such that the guidewire 108 moves over the rigid member 110, under the rigid member 110, or through the through hole of the rigid member 110 when such a through hole is present, the guidewire 108 being in sufficient contact with the rigid member 110 to provide the user-perceptible vibrations in the guidewire 108 or the handholdable portion 112 of the medical device 100. Notably, with respect to the guidewire 108 moving over the rigid member 110, the guidewire 108 is between the rigid member 110 and the user of the medical device 100 from a perspective of the user looking down onto the medical device 100 like the top view of the catheter assembly 102 illustrated in FIG. 2 or that shown in FIG. 5 by way of the eye symbol. With respect to the guidewire 108 moving under the rigid member 110, the rigid member 110 is between the guidewire 108 and the user of the medical device 100 from a perspective of the user looking down onto the medical device 100. While the guidewire 108 moving under or through the rigid member 110 is not shown, it should be understood that the pathway or channel through which the guidewire 108 moves in the medical device 100, for example, the catheter assembly 102 or the guidewire-management device 106, can be modified from that respectively shown in FIGS. 1-4 and FIGS. 5 and 6 to be under or through the rigid member 110.

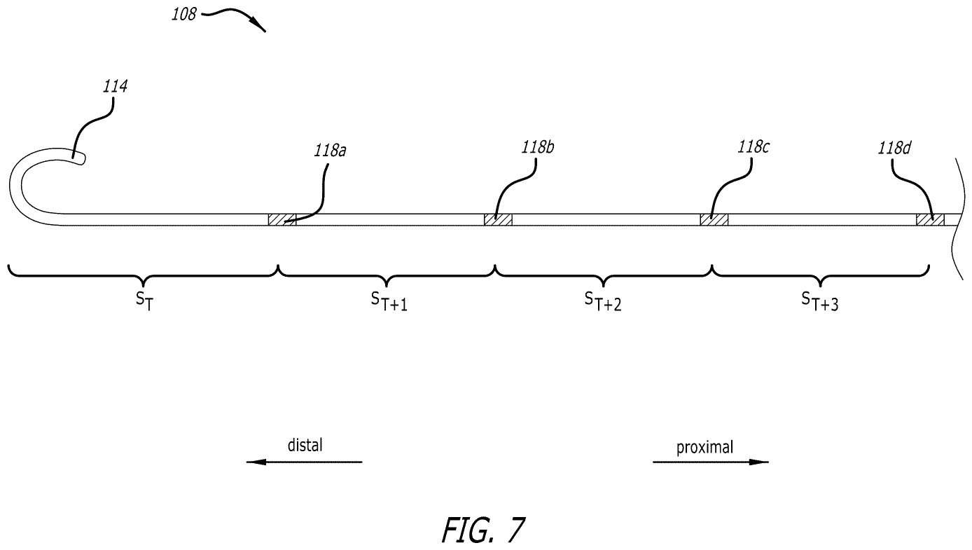

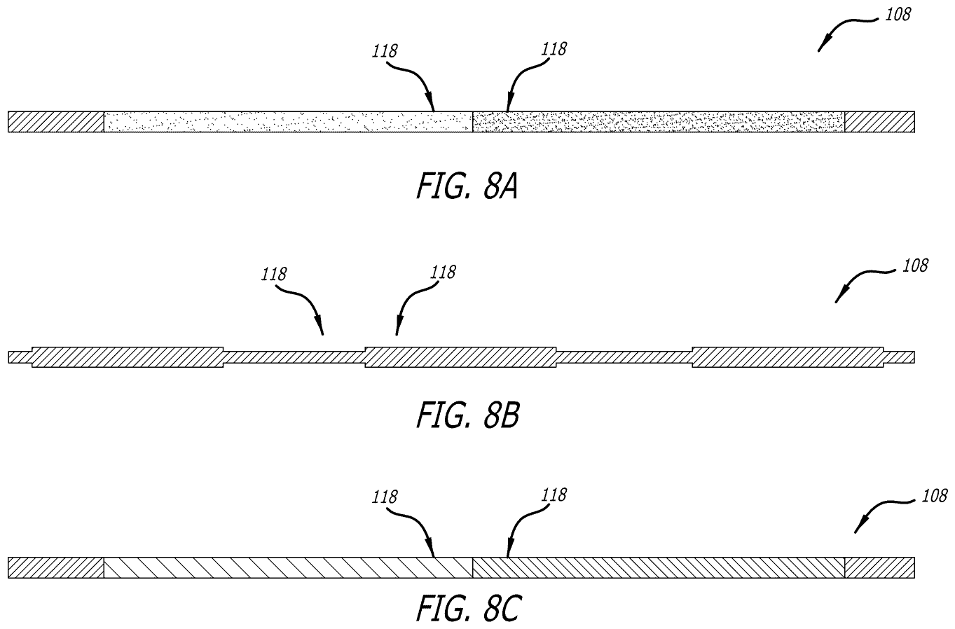

FIG. 7 illustrates a detailed view of a generic version of the guidewire 108 including interactive features 118 in accordance with some embodiments. FIGS. 8A-8C illustrate detailed views of different interactive features 118 in accordance with some embodiments.

As shown, the guidewire 108 includes interactive features 118a, 118b, 118c, . . . 118n, collectively 118, along its length that interact with the rigid member 110 to indicate the status of the guidewire 108 with respect to the medical device 100 or a patient upon which the medical device 100 is being used. The interactive features 118 can demarcate different segments of the guidewire 108 including a tip segment S_{T}, a first segment S_{T+1} proximal of the tip segment S_{T}, a second segment S_{T+2} proximal of the first segment S_{T+1}, a third segment S_{T+3} proximal of the second segment S_{T+2}, and so on, which segments are of similar lengths, different lengths, or a mixture thereof. With knowledge of the interactive features 118, optionally, the different segments demarcated thereby, and, further optionally, a length of the pathway or channel through which the guidewire 108 moves in the medical device 100, a user of the medical device 100 with the tactile-feedback system 104 is able to know the status of the guidewire 108 with respect to the medical device 100 or a patient upon which the medical device 100 is being used when advancing the guidewire 108 out of the medical device 100 or retracting the guidewire 108 into the medical device 100.

By way of example, the interactive features 118 can demarcate different segments of the guidewire 108 in which the tip segment S_{T} includes a pigtail extending from a zeroth or distalmost length (e.g., ≤10 cm) of the guidewire 108, the first segment S_{T+1} includes a first 10-cm length of the guidewire 108 extending from the tip segment S_{T}, the second segment S_{T+2} includes a second 10-cm length of the guidewire 108 extending from the first segment S_{T+1}, the third segment S_{T+3} includes a third 10-cm length of the guidewire 108 extending from the second segment S_{T+2}, and so on. If the tip segment S_{T} of the guidewire 108 at a total straightened length of 10 cm is wholly disposed in the medical device 100 and the interactive feature 118a is aligned with the rigid member 110, then advancement of the guidewire 108 from the medical device 100 until a first instance of tactile feedback is perceived via the interactive feature 118b indicates 10 cm of the guidewire 108 has advanced out of the medical device 100 and, optionally, into a patient if the medical device 100 is being used on the patient. Advancement of the guidewire 108 until a second instance of tactile feedback is perceived via the interactive feature 118c indicates another 10 cm of the guidewire 108 has advanced out of the medical device 100 and, optionally, into the patient. Likewise, retraction of the guidewire 108 from the interactive feature 118c until a first instance of tactile feedback is perceived via the interactive feature 118b indicates 10 cm of the guidewire 108 has retracted into the medical device 100. Retraction of the guidewire 108 until a second instance of tactile feedback is perceived via the interactive feature 118a indicates another 10 cm of the guidewire 108 has retracted into the medical device 100. In accordance with the foregoing example, a user of a medical device 100 with the tactile-feedback system 104 is able to know the status of the guidewire 108 with respect to the medical device 100 or a patient upon which the medical device 100 is being used when advancing the guidewire 108 out of the medical device 100 or retracting the guidewire 108 into the medical device 100.

Notwithstanding the foregoing, it should be understood that the interactive features 118 can demarcate different segments of the guidewire 108 having similar lengths, different lengths, or a mixture thereof, as set forth above, which depends, at least in part, upon a structure of the medical device 100. In an example of the latter, the tip segment S_{T} of the guidewire 108 includes the pigtail extending from a 10-cm zeroth or distalmost length of the guidewire 108 for a total straightened length of >10 cm (e.g., 15 cm) for the tip segment S_{T}, the first segment S_{T+1} of the guidewire 108 has a commensurate length of >10 cm (e.g., 15 cm), and each successive segment of the guidewire 108 proximal of the first segment has a length of 10 cm. If the tip segment S_{T} of the guidewire 108 at a total straightened length of >10 cm (e.g., 15 cm) is wholly disposed in the medical device 100 and the interactive feature 118a is aligned with the rigid member 110, then advancement of the guidewire 108 from the medical device 100 until a first instance of tactile feedback is perceived via the interactive feature 118b indicates a commensurate >10 cm (e.g., 15 cm) of the guidewire 108 has advanced out of the medical device 100 and, optionally, into a patient if the medical device 100 is being used on the patient. Advancement of the guidewire 108 until a second instance of tactile feedback is perceived via the interactive feature 118c indicates a further 10 cm of the guidewire 108 has advanced out of the medical device 100 and, optionally, into the patient. Likewise, retraction of the guidewire 108 from the interactive feature 118c until a first instance of tactile feedback is perceived via the interactive feature 118b indicates 10 cm of the guidewire 108 has retracted into the medical device 100. Retraction of the guidewire 108 until a second instance of tactile feedback is perceived via the interactive feature 118a indicates a further >10 cm (e.g., 15 cm) of the guidewire 108 has retracted into the medical device 100. Accordingly, it should be understood that the segments S_{T}, S_{T+1}, S_{T+2}, . . . , S_{T+n} of the guidewire 108 can vary in length, as appropriate, per the medical device 100 and the pathway or channel into which the guidewire 108 is incorporated.

As to the interactive features 118 of the guidewire 108, themselves, each interactive feature 118 of the guidewire 108 includes a single transitional feature or a plurality of transitional features between one segment of the segments S_{T}, S_{T+1}, S_{T+2}, . . . , S_{T+n} of the guidewire 108 and a next segment thereof that allows for user differentiation of the foregoing segments in accordance with the corresponding tactile feedback. A single transitional feature can include an instant transition from one segment of the guidewire 108 to a next segment thereof when the foregoing segments differ with respect to guidewire diameter as shown in FIG. 8B, surface features, surface characteristics such as surface roughness as shown in FIG. 8A, composition, construction such as winding-wire windings around a core wire as shown in FIG. 8C, or some combination thereof. Alternatively, a single transitional feature can include an applied feature applied to the guidewire 108 between one segment of the guidewire 108 and a next segment thereof whether or not the foregoing segments differ with respect to guidewire diameter, surface features, surface characteristics, composition, construction, or some combination thereof. Likewise, a plurality of transitional features can include a combination or pattern of applied features applied to the guidewire 108 between one segment of the segments of the guidewire 108 and a next segment thereof whether or not the foregoing segments differ in guidewire diameter, surface features, surface characteristics, composition, construction, or some combination thereof. The applied feature(s) applied to the guidewire 108 between one segment of the guidewire 108 and the next segment thereof exhibit(s) some difference(s) with respect to guidewire diameter, surface features, surface characteristics, composition, construction, or some combination thereof when compared to the foregoing segments.

Given that the interactive features 118 of the guidewire 108 include transitional features utilizing differences in guidewire diameter, surface features, surface characteristics, composition, construction, or some combination thereof, a number of possibilities exist for indicating the status of the guidewire 108 with respect to the patient or the medical device 100 by way of the tactile-feedback system 104. In an example, the guidewire diameter can change from one of a larger diameter or a smaller diameter to the other of the larger diameter or the smaller diameter as shown in FIG. 8B to indicate the status of the guidewire 108 with respect to the patient or the medical device 100. In another example, the guidewire surface features can change from one of a presence or a lack of the guidewire surface features to the other of the presence or the lack of the guidewire surface features to indicate the status of the guidewire 108 with respect to the patient or the medical device 100. Such guidewire surface features can include ridges, grooves, or some combination thereof resulting from a relatively loose winding pitch of a wound wire over a core wire in one or more wound segments of the guidewire 108. In another example, the guidewire surface characteristics can include surface roughness that changes from one degree of the surface roughness to another degree of the surface roughness as shown in FIG. 8A to indicate the status of the guidewire 108 with respect to the patient or the medical device 100. In another example, the guidewire construction can change from one of a bare-wire portion, a round-wound construction, a ground-wound construction, or a flat-wound potion of the guidewire 108 to another of the bare-wire portion, the round-wound construction, the ground-wound construction, or the flat-wound potion of the guidewire 108 to indicate the status of the guidewire 108 with respect to the patient or the medical device 100. In another example, the guidewire construction can change from one of a round-wound construction, a ground-wound construction, or a flat-wound potion of the guidewire 108 to another construction of the same but having a different winding pitch of the wound wire over the core wire as shown in FIG. 8C to indicate the status of the guidewire 108 with respect to the patient or the medical device 100.

FIG. 9 illustrates a detailed view of the guidewire 108 including a particular set of the interactive features 118 in accordance with some embodiments.

As shown, the guidewire 108 includes the segments S_{T}, S_{T+1}, S_{T+2}, . . . , S_{T+n} of the guidewire 108 and the interactive features 118 interactive features 118a, 118b, 118c, . . . 118n, collectively 118, along its length, which interactive features 118 interact with the rigid member 110 to indicate the status of the guidewire 108 with respect to the medical device 100 or a patient upon which the medical device 100 is being used.

Beginning with a base structure and composition of the guidewire 108, which can be applied to any guidewire herein, the guidewire 108 can include a core wire and one or more wound or braided wires over the core wire, each of which wires are independently selected from stainless steel and nitinol. Further to the structure of the guidewire 108, the distal tip 114 in a distal portion of the guidewire 108 can be a straight tip, the ‘J’-shaped tip, as shown, or a helically coiled tip.

Continuing with the segments of the guidewire 108, the segment Sr includes a proximal portion up to an entirety of the segment Sr having a finish in which surface oxides are removed, for example, by laser ablation or grinding. Additionally or alternatively, the proximal portion up to the entirety of the segment S_{T} has a coating. The coating can be a hydrophobic coating including a polyurethane coating, a fluoropolymer coating such as that of polytetrafluoroethylene (“PTFE”), a perfluoroalkoxy alkane (“PA”), or fluorinated ethylene propylene (“FEP”), a parylene coating, a polyethylene coating, a polyetheretherketone (“PEEK”) coating, a silicone coating, or a siloxane-based coating such as that of polydimethylsiloxane (“PDMS”). Alternatively, the coating can be a hydrophilic coating including a polyvinylpyrrolidone (“PVP”) coating, a polyethylene glycol (“PEG”) coating, a hydrogel coating, or a polysaccharide-based coating such as that of hyaluronic acid (“HA”). As to each segment of the guidewire 108 following the segment Sr in a proximal direction, namely, each segment of segments S_{T+1}, S_{T+2}, . . . , S_{T+n} of the guidewire 108, each segment can have the foregoing base structure and composition of the guidewire 108. Notably, the guidewire 108 surface of each segment of the segments S_{T+1}, S_{T+2}, . . . , S_{T+n} of the guidewire 108 has a surface roughness characteristic of the surface oxides thereon, thereby having a greater surface roughness than the segment S_{T} of the guidewire 108.

Continuing with the interactive features 118 along the length of the guidewire 108, the interactive feature 118a is a single transitional feature like that set forth above in which an instant transition from the segment S_{T} of the guidewire 108 to the segment S_{T+1} of the guidewire 108 allows for user differentiation of the foregoing segments in accordance with the corresponding tactile feedback. Being that each segment of segments S_{T+1}, S_{T+2}, . . . , S_{T+n} of the guidewire 108 has the foregoing base structure and composition of the guidewire 108, however, the interactive features 118a, 118b, 118c, . . . 118n are applied features applied to the guidewire 108 such that the interactive features 118a, 118b, 118c, . . . 118n intervene with the segments S_{T+1}, S_{T+2}, . . . , S_{T+n} of the guidewire 108 beginning with the interactive feature 118b intervening between the segments S_{T+1} and S_{T+2} of the guidewire 108, as shown, thereby allowing for user differentiation between flanking segments of the guidewire 108 by way of the interactive features 118 and their tactile feedback. Like that set forth above, the applied features applied to the guidewire 108 can exhibit differences from flanking segments of the guidewire 108, those differences including at least guidewire surface characteristics such as having a lesser surface roughness than the flanking segments, for example, by removal of the surface oxides or application of a coating, or a greater surface roughness than the flanking segments, for example, by mechanical or chemical surface texturing, the mechanical surface texturing including, but not limited to, grinding, sanding, media blasting, scoring, or patterning.

As set forth above, when the interactive features 118 are applied features, each applied feature can be a single applied feature or a combination or pattern of applied features applied to the guidewire 108 for user differentiation from the base structure and composition of the guidewire 108 in accordance with the corresponding tactile feedback. Where a single applied feature as the interactive feature 118 between flanking segments of the guidewire 108 can readily allow a user to differentiate between the foregoing segments by way of the tactile feedback associated therewith, a combination or pattern of applied features applied to the guidewire 108 can advantageously provide additional information, thereby obviating or lessening mental tracking of the tactile feedback by a user for understanding a status of the guidewire 108 with respect to a patient or the medical device 100. Focusing on surface roughness for expository expediency, but with extension to other interacting means for interacting with the guidewire 108 disclosed herein, such applied features can include a combination or pattern including different surface roughnesses, lengths of such surface roughnesses, spacings between such surface roughnesses, and sequences thereof, which sequences can include repeating sequences like the repeating sequence of singlets and doublets of applied features shown in FIG. 10.

In an example, the interactive features 118 of the guidewire 108 shown in FIG. 9 include the interactive feature 118b, which includes a singlet or single applied feature of a lesser or greater surface roughness than its flanking segments of the guidewire 108, the interactive feature 118c, which includes a combination of applied features for a spaced doublet of applied features of lesser or greater surface roughness than its flanking segments of the guidewire 108, the interactive feature 118d, which includes a combination of applied features for a spaced triplet of applied features of lesser or greater surface roughness than its flanking segments of the guidewire 108, and so on. The foregoing interactive features 118 thereby provide a pattern of applied features, wherein the singlet or single applied feature corresponding to the interactive feature 118b can indicate an initial unit of guidewire length (e.g., 10 cm) has advanced out of the medical device 100 and, optionally, into a patient, the doublet of applied features corresponding to the interactive feature 118c can indicate a second unit of guidewire length (e.g., another 10 cm) has advanced out of the medical device 100 and, optionally, into the patient, the triplet of applied features corresponding to the interactive feature 118d can indicate a third unit of guidewire length (e.g., yet another 10 cm) has advanced out of the medical device 100 and, optionally, into the patient, and so on. As such, a combination or pattern of applied features applied to the guidewire 108 can advantageously provide additional information, thereby obviating or lessening mental tracking of the tactile feedback by the user for understanding the status of the guidewire 108 with respect to the patient or the medical device 100.

FIG. 10 illustrates a detailed view of the guidewire 108 including another plurality of the interactive features 118 in accordance with some embodiments.

Following on the embodiment of the guidewire 108 shown in FIG. 9, the guidewire 108 shown in FIG. 10 includes the interactive feature 118a as an applied feature applied to the guidewire 108 such that the interactive feature 118a intervenes between the segments S_{T} and S_{T+1} of the guidewire 108, as shown, thereby allowing for greater contrast than any instant transition between the segment S_{T} of the guidewire 108 to the segment S_{T+1} of the guidewire 108 described in reference to FIG. 9. This can result in enhanced user differentiation of the foregoing segments. Indeed, in an example, such an applied feature can have a lesser surface roughness than both its flanking segments S_{T} and S_{T+1} of the guidewire 108 or a greater surface roughness than the flanking segments, which surface roughness can occur over a longer length of the guidewire 108 than the instant transition between the segments S_{T} and S_{T+1} of the guidewire 108 for the enhanced user differentiation. Advantageously, the foregoing interactive feature 118a can include any differences in guidewire diameter, surface features, surface characteristics, composition, or construction set forth herein to provide for the enhanced user differentiation, which is useful for indicating to a user of the medical device 100 that the segment S_{T} of the guidewire 108 remains in the medical device 100 or is being positioned therein.

Further to the guidewire 108 shown in FIG. 10, the interactive feature 118b includes a singlet or single applied feature of a lesser or greater surface roughness than its flanking segments of the guidewire 108, the interactive feature 118c includes a spaced doublet of applied features of lesser or greater surface roughness than its flanking segments of the guidewire 108, the interactive feature 118d includes another singlet or single applied feature of a lesser or greater surface roughness than its flanking segments of the guidewire 108, and so on for alternating singlets and doublets of applied features of lesser or greater surface roughness than their flanking segments of the guidewire 108. The foregoing interactive features 118 thereby provide a pattern of applied features, wherein the singlet or single applied feature corresponding to the interactive feature 118b can indicate an initial unit of guidewire length (e.g., 10 cm) has advanced out of the medical device 100 and, optionally, into a patient, the doublet of applied features corresponding to the interactive feature 118c can indicate a second unit of guidewire length (e.g., another 10 cm) has advanced out of the medical device 100 and, optionally, into the patient, the singlet or single applied feature corresponding to the interactive feature 118d can indicate a third unit of guidewire length (e.g., yet another 10 cm) has advanced out of the medical device 100 and, optionally, into the patient, and so on. As such, a combination or pattern of applied features applied to the guidewire 108 can advantageously provide additional information, thereby obviating or lessening mental tracking of the tactile feedback by the user for understanding the status of the guidewire 108 with respect to the patient or the medical device 100.

While the guidewire 108 and the combinations or patterns of applied features shown in FIGS. 9 and 10 for the interactive features 118 consist of singlets or multiplets of different surface roughnesses than their flanking segments of the guidewire 108, it should be understood that the foregoing singlets and multiplets of different surface roughnesses provide but one example for encoding guidewire extension from the medical device 100 or depth into a patient into the guidewire 108. In an alternative example, instead of alternating the singlets and doublets of surface-roughness-based applied features corresponding to the interactive features 118 of the guidewire 108 of FIG. 10, the applied features can alternate from an applied feature of lesser surface roughness than its flanking segments, to an applied feature of greater surface roughness than its flanking segments, to another applied feature of lesser surface roughness than its flanking segments, and so on to encode guidewire extension from the medical device 100 or depth into a patient into the guidewire 108.

In another example, instead of the singlet, doublet, triplet, and so on of surface-roughness-based applied features corresponding to the interactive features 118 of the guidewire 108 of FIG. 9, the applied features can resemble International Morse Code, which is incorporated herein by reference in its entirety, in that binary sequences of applied features of different surface roughnesses than the guidewire 108 can encode guidewire extension from the medical device 100 or depth into a patient into the guidewire 108. Indeed, an applied feature of a lesser surface roughness than its flanking segments of the guidewire 108 can indicate a “dot”, and an applied feature of a greater surface roughness than its flanking segments of the guidewire 108 can indicate a “dash,” thereby providing a binary system for International Morse Code or a simplified version thereof for the medical device 100 in which, for example, a digit encoded in International Morse Code is a multiplier for a particular unit length of the guidewire 108. Alternatively, an applied feature of a relatively short length of lesser or greater surface roughness than its flanking segments of the guidewire 108 can indicate a “dot”, and an applied feature of a relatively long length of the lesser or greater surface roughness than its flanking segments of the guidewire 108 can indicate a “dash,” thereby providing another binary system for International Morse Code or a simplified version thereof for the medical device 100 in which, for example, a digit encoded in International Morse Code is a multiplier for a particular unit length of the guidewire 108. However, it should be understood, as above, that the applied features can exhibit any difference with respect to guidewire diameter, surface features, surface characteristics, composition, construction, or some combination thereof when compared to their flanking segments. As such, any description set forth herein with respect to applied features being different with respect to guidewire surface characteristics such as surface roughness can be extended to guidewire diameter, surface features, other surface characteristics, composition, construction, or some combination thereof.

### Catheter Assembly

Again, FIGS. 1-4 illustrate the catheter assembly 102 as the medical device 100 including the tactile-feedback system 104 in accordance with some embodiments.

As shown, the catheter assembly 102 can include an introducer needle 120, a coupler 122, a catheter 124, and the guidewire 108 assembled together, optionally, with a syringe 126 coupled to the introducer needle 120, in a ready-to-deploy state of the catheter assembly 102.

With a focus on the tactile-feedback system 104 of the catheter assembly 102, the coupler 122 includes a coupler housing 128 and a valve module 130 disposed therein. Indeed, the valve module 130 is disposed in the coupler housing 128, and, as shown, both a composite shaft 132 (e.g., an introducer sheath over a slotted needle) of the introducer needle 120 and the guidewire 108 pass through the valve module 130 in the ready-to-deploy state of the catheter assembly 102 such that the valve module 130 forms seals over both the composite shaft 132 and the guidewire 108. The rigid member 110 is disposed in the coupler 122 such that it transversely extends from one side of the coupler housing 128 to another side of the coupler housing 128 proximal of the valve module 130. In addition, the pathway or channel of the catheter assembly 102 through which the guidewire 108 moves extends from an introducer-needle lumen of the introducer needle 120, through the valve module 130, and over the rigid member 110. Configured as such, the rigid member 110 is in vibratory communication with a pair of grips, as shown in FIG. 2, distributed between the same sides of the coupler housing 128 in which the rigid member 110 is disposed, thereby efficiently producing the user-perceptible vibrations in the grips of the coupler 122 or coupler housing 128 thereof as the guidewire 108 moves against the rigid member 110 while the guidewire 108 is being advanced out of the catheter assembly 102 or retracted into the catheter assembly 102.

Notably, while the ‘J’-shaped distal tip 114 of the guidewire 108 is shown, the distal portion of the guidewire 108 is retracted in the ready-to-deploy state of the catheter assembly 102 such that the interactive feature 118a is in contact with the rigid member 110, just proximal of the rigid member 110, or just distal of the rigid member 110, thereby placing the distal tip 114 of the guidewire 108 in the introducer-needle lumen just proximal of the needle tip of the introducer needle 120. In this way, the distal tip 114 of the guidewire 108 can be immediately available to advance into a blood vessel lumen of a patient after a venipuncture with the introducer needle 120 up to depth corresponding to the interactive feature 118b, 118b, . . . , 118n.

### Guidewire-Management Device

Again, FIGS. 5 and 6 illustrate the guidewire-management device 106 as the medical device 100 including the tactile-feedback system 104 in accordance with some embodiments.

As shown, the guidewire-management device 106 can include an elongate body 134 having a distal sleeve 136 formed in a distal portion of the body 134, a proximal sleeve 138 formed in a proximal portion of the body 134, and a gapped guidewire conduit 140 coupled to the proximal sleeve 138 and a handle of the body 134, as shown in FIG. 5, the gapped guidewire conduit 140 having a gap for advancing or retracting the guidewire 108 by hand.

With a focus on the tactile-feedback system 104 of the guidewire-management device 106, the body 134 of the guidewire-management device 106 can be considered commensurate with a housing of the guidewire-management device 106 being that the body 134 of the guidewire-management device 106 is typically a unitary molded component. Disposed in the body 134 of the guidewire-management device 106 in line with the proximal sleeve 138 and the distal sleeve 136 but longitudinally therebetween is a guidewire support 142. Disposed in the guidewire support 142, in turn, is the rigid member 110. Indeed, the rigid member 110 is disposed in the guidewire support 142 such that it transversely extends from one side to another side of the guidewire-management device 106, which rigid member 110, as shown, is perpendicular to the proximal sleeve 138 and the distal sleeve 136. The pathway or channel of the guidewire-management device 106 through which the guidewire 108 moves thusly extends from the guidewire conduit 140, through the proximal sleeve 138, over the rigid member 110 in the guidewire support 142, through the distal sleeve 136, and out the guidewire-management device 106. Configured as such, the rigid member 110 in the guidewire support 142 is in vibratory communication with the handle, thereby efficiently producing the user-perceptible vibrations in the handle as the guidewire 108 moves against the rigid member 110 while the guidewire 108 is being advanced out of the guidewire-management device 106 or retracted into the guidewire-management device 106.

Notably, while the ‘J’-shaped distal tip 114 of the guidewire 108 is shown, the distal portion of the guidewire 108 is retracted in a ready-to-deploy state of the guidewire-management device 106 such that the interactive feature 118a is in contact with the rigid member 110, just proximal of the rigid member 110, or just distal of the rigid member 110, thereby placing the distal tip 114 of the guidewire 108 in the distal sleeve 136 just proximal of a sleeve tip of the distal sleeve 136. In this way, the distal tip 114 of the guidewire 108 can be immediately available to advance into a blood vessel lumen of a patient after a venipuncture with the introducer needle 120 up to depth corresponding to the interactive feature 118b, 118b, 118n.

Advantages of such guidewire-containing medical devices having the tactile-feedback system 104 include providing sensory information to a user for understanding the status of the guidewire 108 with respect to a patient or its corresponding medical device 100 that does not depend on vision. Indeed, while some guidewires include visual markings to provide visual information regarding the guidewires, such visual markings can be difficult to visualize due to poor lighting, low contrast, small guidewire diameter, or obfuscation by blood or other substances during use. Further, there is also a tendency for the user to look away from the guidewire or its corresponding medical device when focusing on imaging, the vascular insertion site, or the like. That, and including visual markings on such guidewires can be costly or infeasible unlike implementation of the tactile-feedback system 104 in the medical device 100.

### Methods

Methods include making and using guidewire-containing medical devices with the tactile-feedback system 104.

In an example of using the guidewire-containing medical device 100 with the tactile-feedback system 104, such a method can include one or more operations selected from at least obtaining the medical device 100, advancing the guidewire 108 out of the medical device 100, and retracting the guidewire 108 into the medical device 100. As set forth above, the tactile-feedback system 104 includes the guidewire 108 and the rigid member 110 in vibratory communication with the handholdable portion 112 of the medical device 100. The guidewire 108 includes the interactive features 118 along its length. The interactive features 118 of the guidewire 108 interact with the rigid member 110 to produce user-perceptible vibrations in the guidewire 108 or the handholdable portion 112 of the medical device 100 as the guidewire 108 moves against the rigid member 110. Thus, advancing the guidewire 108 out of the medical device 100 or retracting the guidewire 108 into the medical device 100 moves the guidewire 108 over the rigid member 110, thereby providing tactile feedback to a user of the medical device 100 indicating a status of the guidewire 108 with respect to a patient or the medical device 100.

In an example of making the guidewire-containing medical device 100 with the tactile-feedback system 104, such a method can include one or more operations selected from at least making the guidewire 108 and disposing the rigid member 110 in the medical device 100 such that it is in vibratory communication with the handholdable portion 112 of the medical device 100. As to making the guidewire 108, making the guidewire 108 can include varying its structure such as its diameter or its composition while establishing the base structure and composition of the guidewire 108, thereby providing the guidewire 108 with at least some interactive features 118. However, it should be understood that establishing the base structure and composition of the guidewire 108 need not include providing any of the interactive features 118. Indeed, making the guidewire 108 can additionally or alternatively include a separate interactive-feature-establishing operation in which the interactive features 118 are established by removing surface oxides by laser ablation, grinding, sanding, or media blasting, coating the guidewire 108 with one or more coatings, mechanical or chemical surface texturing, or some combination thereof. Notably, the mechanical surface texturing can include, but is not limited to, grinding, sanding, media blasting, scoring, or patterning.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. A tactile-feedback system for a guidewire-containing medical device, comprising:
a guidewire including interactive features along its length; and
a rigid member in vibratory communication with a handholdable portion of the medical device, the interactive features of the guidewire interacting with the rigid member to produce user-perceptible vibrations in the guidewire or the handholdable portion of the medical device as the guidewire moves against the rigid member while being advanced out of the medical device or retracted into the device, thereby providing tactile feedback to a user of the medical device indicating a status of the guidewire with respect to a patient or the medical device.

2. The tactile-feedback system of claim 1, wherein the guidewire moves over the rigid member, the guidewire being between the rigid member and the user of the medical device from a perspective of the user looking down onto the medical device.

3. The tactile-feedback system of claim 1, wherein the guidewire moves under the rigid member, the rigid member being between the guidewire and the user of the medical device from a perspective of the user looking down onto the medical device.

4. The tactile-feedback system of claim 1, wherein the guidewire moves through a through hole of the rigid member.

5. The tactile-feedback system of claim 1, wherein the rigid member is a metal insert disposed in a housing of the medical device or a structural component associated with the housing.

6. The tactile-feedback system of claim 5, wherein the rigid member is a metal rod or plate.

7. The tactile-feedback system of claim 1, wherein the status of the guidewire with respect to the patient is a depth to which the guidewire is inserted into the patient.

8. The tactile-feedback system of claim 1, wherein the status of the guidewire with respect to the medical device is whether a distal tip of the guidewire is within the medical device or outside the medical device.

9. The tactile-feedback system of claim 1, wherein the interactive features of the guidewire include differences in guidewire diameter, surface features, surface characteristics, composition, construction, or some combination thereof.

10. The tactile-feedback system of claim 9, wherein the guidewire diameter changes from one of a larger diameter or a smaller diameter to the other of the larger diameter or the smaller diameter to indicate the status of the guidewire with respect to the patient or the medical device.

11. The tactile-feedback system of claim 9, wherein the guidewire surface features change from one of a presence or a lack of the guidewire surface features to the other of the presence or the lack of the guidewire surface features to indicate the status of the guidewire with respect to the patient or the medical device.

12. The tactile-feedback system of claim 11, wherein the guidewire surface features are ridges, grooves, or some combination thereof.

13. The tactile-feedback system of claim 9, wherein the guidewire surface characteristics include surface roughness that changes from one degree of the surface roughness to another degree of the surface roughness to indicate the status of the guidewire with respect to the patient or the medical device.

14. The tactile-feedback system of claim 9, wherein the guidewire construction changes from one of a bare-wire portion, a round-wound construction, a ground-wound construction, or a flat-wound potion of the guidewire to another of the bare-wire portion, the round-wound construction, the ground-wound construction, or the flat-wound potion of the guidewire to indicate the status of the guidewire with respect to the patient or the medical device.

15. The tactile-feedback system of claim 1, wherein the medical device is a catheter assembly.

16. The tactile-feedback system of claim 1, wherein the medical device is a guidewire-management device.

17. A method of using a guidewire-containing medical device with a tactile-feedback system, comprising:
obtaining the medical device, the tactile-feedback system including:
a guidewire including interactive features along its length;
a rigid member in vibratory communication with a handholdable portion of the medical device, the interactive features of the guidewire interacting with the rigid member to produce user-perceptible vibrations in the handholdable portion of the medical device as the guidewire moves against the rigid member; and
advancing the guidewire out of the medical device or retracting the guidewire into the medical device, thereby providing tactile feedback to a user of the medical device indicating a status of the guidewire with respect to a patient or the medical device.

18. The method of claim 17, wherein advancing the guidewire out of the medical device or retracting the guidewire into the medical device moves the guidewire over the rigid member, the guidewire being between the rigid member and the user of the medical device from a perspective of the user looking down onto the medical device.

19. The method of claim 17, wherein the rigid member is a metal rod or plate disposed in a housing of the medical device or a structural component associated with the housing.

20. The method of claim 19, wherein the status of the guidewire with respect to the patient is a depth to which the guidewire is inserted into the patient, and the status of the guidewire with respect to the medical device is whether a distal tip of the guidewire is within the medical device or outside the medical device.

21. The method of claim 17, wherein the interactive feature of the guidewire include differences in guidewire diameter, surface features, surface characteristics, composition, construction, or some combination thereof.
